(19) United States
(12) Patent Application Publication
(10) Pub. No.: US 2013/0084586 A1
(43) Pub. Date: Apr. 4, 2013

(54) RAPID, SPECIFIC AND SENSITIVE IMMUNOASSAYS FOR THE DETECTION OF HIGHLY VARIABLE GRAM NEGATIVE BACTERIAL ANTIGENS

(71) Applicant: The Penn State Research Foundation, University Park, PA (US)

(72) Inventors: NARASIMHA HEGDE, State College, PA (US); Chitrita Debroy, State College, PA (US); Vivek Kapur, Port Matilda, PA (US)

(73) Assignee: The Penn State Research Foundation, University Park, PA (US)

(21) Appl. No.: 13/630,948

(22) Filed: Sep. 28, 2012

Related U.S. Application Data
(60) Provisional application No. 61/541,554, filed on Sep. 30, 2011.

(51) Int. Cl. G01N 33/68

(52) U.S. Cl. CPC G01N 33/6893
USPC 435/7.93; 436/501; 530/350; 435/7.92; 435/7.94

(57) ABSTRACT

Methods for the detection, identification and quantification of gram negative bacteria and gram negative bacterial antigens are rapid, efficient and highly specific. Compositions for the detection and identification of highly variable serogroups are provided.

### CROSS-REFERENCE TO RELATED APPLICATIONS

This application claims priority to U.S. provisional patent application No. 61/541,554 entitled “RAPID, SPECIFIC AND SENSITIVE IMMUNOASSAYS FOR THE DETECTION OF HIGHLY VARIABLE GRAM NEGATIVE BACTERIAL ANTIGENS”, filed Sep. 30, 2011, which is incorporated herein by reference in its entirety.

### FIELD OF THE INVENTION

Embodiments of the invention provide methods for the rapid and sensitive detection of gram negative bacterial antigens.

### BACKGROUND

The Centers for Disease Control and Prevention (CDC) has estimated that non-O157 shiga toxin producing Escherichia coli (STEC) are responsible for 36,000 illnesses each year in the United States (CDC, 2008. Morb. Mortal. Wkly. Rep. 57:366-370). The majority of these infections have been associated with six serogroups: O26, O45, O103, O111, O121, and O145 (Brooks et al. 2005, J. Infect Dis. 192:190-198). Since the reporting of all STEC illnesses to the CDC began in 2001, instances of non-O157 STEC illnesses have steadily increased and there have been 30 major documented outbreaks associated with non-O157 STEC serotypes in the United States in the past 25 years (CDC, 2008. Morb. Mortal. Wkly. Rep. 57:366-370). It is believed that non-O157 STEC may cause diarrhea at frequencies similar to those important enteric pathogens such as Salmonella and Shigella (Slutsker et al. 1997. Ann. Intern. Med. 126: 505-513) resulting in hemorrhagic colitis (HC) and hemolytic uremic syndrome (HUS) (Karmali et al. 2003. J. Clin. Microbiol. 41: 4930-4940). One of the primary reasons for the underreporting of non-O157 STEC serogroups associated with illnesses is because of difficulties confirming their serotypes.

### SUMMARY

Embodiments of the invention comprise a rapid, sensitive and specific assay for the detection of gram negative bacterial antigens with diverse serogroups. The assay has wide applicability in the field for detection of organisms that are responsible for human and veterinary illness and death if not diagnosed and treated in time.

In embodiments, a method of detecting and quantifying variable antigens in a biological sample comprises contacting a biological assay substrate with a first agent which specifically binds to epitopes in a conserved region of an antigen wherein the antigen comprises conserved and variable regions; adding the antigen to the biological assay device wherein the agent specifically binds to the epitopes in the conserved region of the antigen; adding a second agent which specifically binds to the variable region of the antigen. In some embodiments, the antigen is isolated from gram negative bacteria. In another embodiment, the antigen is isolated from a gram positive bacterium. In other embodiments, the antigen is synthetic. In yet other embodiments, the antigen is isolated from an organism comprising: a virus, a parasite, bacteria, a fungus, mammals, and the like. In some embodiments, the antigen comprises a conserved region of lipid A and a variable region isolated from gram negative bacteria. In some embodiments, the order of adding the antigen to be tested, the agents to be added, can be varied. Thus, in some embodiments, the antigen to be tested is added first and the agents added either at the same time, or added sequentially. For example, the first agent is added and the second agent is then added, with wash steps between each step. In other embodiments, one or more washing steps are omitted.

In embodiments, the concentration of detectable antigen is at least about 0.0001 CFU/ml.

In other embodiments, the second agent comprises a detectable label.

In other embodiments, the first and second agents comprise: antibodies or fragments thereof, aptamers, scaffold peptides, nucleotides; nucleic acids; PNA (peptide nucleic acids); proteins; peptides; carbohydrates; artificial polymers; synthetic or natural molecules, organic or inorganic molecules.

In other embodiments, the antibodies comprise: polyclonal, monoclonal, synthetic, an Fab fragment, an F(ab′)₂ fragment, an Fd fragment, an Fv fragment, a dAb fragment, an isolated complementarity determining region (CDR), a single chain antibody (scFv), or combinations thereof.

In embodiments, the assay comprises immunoassays, lateral flow assays, biochip assays, protein assays, or high-throughput screening assays. In other embodiments, the biological assay substrate comprises tubes, cylinders, beads, discs, silicon chips, microplates, polyvinylidene difluoride (PVDF) membrane, nitrocellulose membrane, nylon membrane, porous membranes, non-porous membranes, plastic, polymer, silicon, polymeric pins, a plurality of microtiter wells, or any combinations thereof. The composition of the substrate can be varied. For example, substrates or support can comprise glass, cellulose-based materials, thermoplastic polymers, such as polyethylene, polypropylene, or polyester, sintered structures composed of particulate materials (e.g., glass or various thermoplastic polymers), or cast membrane film composed of nitrocellulose, nylon, polysulfone, or the like. In general embodiments, the substrate may be any surface or support upon which the first agent can be immobilized, including one or more of a solid support (e.g., glass such as a glass slide or a coated plate, silica, plastic or derivatized plastic, paramagnetic or non-magnetic metal), a semi-solid support (e.g., a polymeric material, a gel, agarose, or other matrix), and/or a porous support (e.g., a filter, a nylon or nitrocellulose membrane or other membrane). In some embodiments, synthetic polymers can be used as a substrate, including, e.g., polystyrene, polypropylene, polyglycidylmethacrylate, aminated or carboxylated polystyrenes, polyacrylamides, polyamides, polyvinylchlorides, and the like. In preferred embodiments, the substrate comprises a microtiter immunoassay plate or other surface suitable for use in an ELISA.

In other embodiments, a composition comprises a chimeric molecule having at least two domains wherein a first domain comprises a predetermined epitope and at least one second domain comprising an antigen wherein the first and second domains are fused or linked. In embodiments, wherein the antigen comprises variable epitopes of gram negative bacterial antigens. The predetermined epitope comprises one or more conserved epitopes.

In other embodiments, the invention provides for a kit for the detection, identification and quantification of bacterial antigens comprising a first agent which specifically binds to a conserved region epitope of the bacterial antigen, a second agent which specifically binds to a variable region epitope of the bacterial antigen.

In other embodiments, a method of detecting and quantifying variable antigens in a biological sample comprising: contacting a biological assay substrate with an antigen wherein the antigen comprises conserved and variable regions, or immobilizing the antigen on surfaces of the biological device; adding an agent which specifically binds to the variable regions of the antigen. In some embodiments, the conserved region of the antigen is immobilized on biological device surfaces, the surfaces comprising: silicon, plastic, glass, polymer, cellulose, polyacrylamide, nylon, polystyrene, polyvinyl chloride or polypropylene, ceramic, photoresist or rubber surface, silicon dioxide or a silicon nitride.

In embodiments wherein the antigen is immobilized on a pre-treated surface, the antigen comprises: a protein, a peptide, an antibody, an artificial protein, an RNA or DNA aptamer, an allosteric ribozyme, nucleic acid, organic or inorganic molecule, carbohydrate or a small molecule.

In another embodiment, a method of detecting and quantifying variable antigens in a biological sample comprising coating a biological assay device with a first agent which specifically binds a predetermined epitope; adding a composition comprising a chimeric molecule having at least two domains wherein a first domain comprises the predetermined epitope and at least one second domain comprising a variable antigenic region to be tested, wherein the first and second domains are fused or linked, and the first agent specifically binds to the epitope of the first domain; adding a second agent which specifically binds to the variable antigenic regions of the second domain. In some embodiments, the first domain comprises: a peptide, oligonucleotide, synthetic molecule, glycoprotein, carbohydrates, organic compounds, inorganic compounds or combinations thereof. In some embodiments, the second domain is an antigen to be tested. In some embodiments, the first domain is conjugated to or attached to the second domain via fusion, a linker molecule, covalent bonds, carbohydrates, or combinations thereof. In some embodiments, the first and second agents comprise: antibodies or fragments thereof, aptamers, scaffolded peptides, nucleotides; nucleic acids; PNA (peptide nucleic acids); proteins; peptides; carbohydrates; artificial polymers; synthetic or natural molecules, organic or inorganic molecules. In other embodiments, the antibodies are polyclonal, monoclonal, synthetic, an Fab fragment, an F(ab′)₂ fragment, an Fd fragment, an Fv fragment, a dAb fragment, an isolated complementarity determining region (CDR), a single chain antibody (scFv), or combinations thereof. In some embodiments, the first agent is immobilized on a biological device surface, the surface comprising: silicon, plastic, glass, polymer, cellulose, polyacrylamide, nylon, polystyrene, polyvinyl chloride or polypropylene, ceramic, photoresist or rubber surface, silicon dioxide or a silicon nitride.

In embodiments, the biological device comprises tubes, beads, discs. silicon chips, microplates, polyvinylidene difluoride (PVDF) membrane, nitrocellulose membrane, nylon membrane, porous membranes, non-porous membranes, plastic, polymer, silicon, polymeric pins, a plurality of microtiter wells, or any combinations thereof.

In other embodiments, the first agent or antigen is immobilized on an optionally pre-treated surface, comprising: a protein, a peptide, an antibody, an artificial protein, an RNA or DNA aptamer, an allosteric ribozyme, nucleic acid, organic or inorganic molecule, carbohydrate or a small molecule.

Other aspects are described infra.

### BRIEF DESCRIPTION OF THE DRAWINGS

FIG. 1 is a schematic representation of an embodiment of a sandwich ELISA for the detection of O antigens. LPS, an abundant cell membrane component of E. coli, is the basis of the ELISAs detection sensitivity. LPS is made up of a lipid A portion, which is conserved throughout the serogroups, a core, and a variable O antigen. Antibodies to the lipid A portion capture the LPS molecules while antibodies to the unique O-antigen allow the specific detection of a serogroup.

FIG. 2 is a graph showing the detection of non-O157 STEC by ELISA. Antigens from respective serogroups were prepared by serial ten-fold dilutions of bacterial cells. Antigens (100 μl) were used in each well coated with anti-lipid A antibody. The mean values of triplicate assays are presented (p<0.001).

FIG. 3 is a graph showing the sensitivity of ELISAs for the detection of STEC O groups in ground beef. Six STEC O groups-O26, O45, O103, O111, O121, and O145-were inoculated in ground beef, enriched overnight, and supernatant used to prepare antigens. ELISAs that produced a signal above the background were considered positive. White bars show the signal produced after enrichment of the target bacteria. Black bars are the background levels seen for the target bacteria when no antigen is present.

FIG. 4 is a graph showing the effect of anti-lipid A antibody on antigen binding. Antigens from the target bacteria were incubated with 5 μg of anti-lipid A (anti-LA) monoclonal antibody for 1 h prior to ELISA. Assays were performed in triplicates and repeated three times (P<0.005). White bars show the absorbance produced with antigen of the target bacteria. Black bars show the reduction in signal production when anti-lipid A is present.

### DETAILED DESCRIPTION

The present invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, “sample” refers to anything which may contain a gram negative bacterial antigen or analyte. The sample may be a food sample, such as, meat, vegetables, fruit, or any ingestible product natural or synthetic. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). The sample may also be a mixture of antigens prepared in vitro.

As used herein, “a control sample” refers to a control sample that is only different in one or more defined aspects relative to a test sample, and the present methods, kits or arrays are used to identify the effects, if any, of these defined difference(s) between the test sample and the control sample. For example, the control sample can be derived from physiological normal conditions and/or can be subjected to different physical, chemical, physiological or drug treatments, or can be derived from different biological stages, etc.

As used herein, “a biological assay device” can be any type of device or format for carrying out an assay. For example, a 96-well plate, microtiter plat, gel, blots, lateral flow immunoassay device, etc. In embodiments, the biological assay devices comprise any device for conducting one or more assays comprising: lateral flow assays, immunoassays, enzyme assays, gels, blots, chromatography, colorimetric, lab-on-a-chip, microfluidics based assays, biochips, microarrays, microchips, nanotube based assays, chromatography, colorimetric assays, spectrophotometric assays or high-throughput screening assays.

The term “contacting” when used in the context of the biological substrate means that the agent e.g. antibody, lipid A, etc., is added to the assay device. This means that if the device is an ELISA plat, the first agent or antigen can be immobilized on the microwell surface, or if it is a bead, the bead is coated with the agent or antigen to be tested, if the substrate is a gel, the first agent or antigen to be tested may be incubated with the gel to allow the for absorption or adsorption. One of ordinary skill in the arts would understand, based on the substrate how best to add the first agent or antigen to be tested.

The term “antibody” as used herein is also meant to include intact molecules as well as fragments thereof, such as, for example, Fab and F(ab′)₂, which are capable of binding the an epitope determinant. As used herein, the term “antibody” encompasses chimeric antibodies and hybrid antibodies, with dual or multiple antigen or epitope specificities, and fragments, such as F(ab′)₂, Fab′, Fab and the like, including hybrid fragments. Thus, fragments of the antibodies that retain the ability to bind their specific antigens are provided. Such antibodies and fragments can be made by techniques known in the art and can be screened for specificity and activity according to general methods for producing antibodies and screening antibodies for specificity and activity (See Harlow and Lane. Antibodies, A Laboratory Manual. Cold Spring Harbor Publications, New York, (1988)).

The term “antigen,” as used herein, refers to a molecule capable of being recognized by an antibody. An antigen can be, for example, a peptide or a modified form thereof. An antigen can comprise one or more epitopes.

The term “epitope,” as used herein, is the part of a macromolecule, such as polypeptides, that is recognized by the immune system, specifically by antibodies, B cells, or cytotoxic T cells. Most epitopes recognized by antibodies can be thought of as three-dimensional surface features of an antigen molecule. These features fit precisely and thus bind to antibodies. These surfaces may depend on tertiary protein structure, such that residues that form an epitope are positioned apart from each other in the amino acid sequence of a protein (conformational epitopes), or may be formed by continuous peptide regions within a protein (linear epitopes). Therefore, if a protein is denatured, as often is the case in diagnostic use of antibodies, only linear epitopes can be used for detection. The number of consecutive amino acid residues that form a linear epitope recognized by an antibody varies, but typically ranges from six to ten (6-10). However, natural antibodies can recognize shorter epitopes with significant affinities, and recombinant antibodies can be targeted to bind even to a single amino acid residue. Thus, an epitope can be a linear epitope, sequential epitope, or a conformational epitope.

As used herein, the term “specifically binding”, or “specifically recognizing”, or the expression “having binding specificity to an epitope” refers to a low background and high affinity binding between a binding moiety (e.g. antibody or fragments thereof) and its target molecule (i.e. lack of non-specific binding). In other words, the terms (and equivalent phrases) refer to the ability of a binding moiety (e.g., a receptor, antibody, ligand or antiligand) to bind preferentially to a particular target molecule (e.g., ligand or antigen) in the presence of a heterogeneous population of proteins and other biologics (i.e., without significant binding to other components present in a test sample). Typically, specific binding between two entities, such as a ligand and a receptor, means a binding affinity of at least about 10⁶ M⁻¹, 10⁷M⁻¹, 10 M⁻¹,10⁹ M⁻¹, or 10¹⁰ M⁻¹, 10¹¹ M⁻¹, 10¹² M⁻¹, 10¹³ M⁻¹, 10¹⁴ M⁻¹, or 10¹⁵ M⁻¹.

The terms “specificity” or “high specificity” may also refer to the capacity of a binding moiety, to bind to 95%, 95.5%, 96%, 96.5%, 97%, 97.5%, 98%, 98.5%, 99%, 99.5% or 100% of the variants of its non-conserved ligand.

The term “identity” means the percentage of amino acid or residues or nucleic acid bases that are identically positioned in a one-dimensional sequence alignment. Identity is a measure of how closely the sequences being compared are related. Identity between two sequences can be determined using the BESTFIT program.

The terms “nucleic acid,” “oligonucleotide” and “polynucleotide” are used interchangeably herein and encompass DNA, RNA, cDNA, whether single stranded or double stranded, as well as chemical modifications thereof.

The terms “nucleic acid,” “oligonucleotide” and “polynucleotide” are used interchangeably herein and encompass DNA, RNA, cDNA, whether single stranded or double stranded, as well as chemical modifications thereof.

The term “linker” means a moiety that connects two chemical components together through either a single covalent bond or multiple covalent bonds.

The terms, “fused” or “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

### Detection of Highly Diverse Antigens

Detection of Highly Diverse Serogroups of Gram Negative Bacteria:

There is a growing concern of potential public health risk associated with non-O157 Shiga toxin-producing Escherichia coli (STEC) with serogroups O26, O45, O103, O111, O121, O145 commonly implicated in outbreaks of human illness worldwide. Current approaches for O serogroup determination are elaborate, extremely labor and resource intensive, requiring from 5 to 9 days to from primary isolation to completion. Hence, the development of new methods for the rapid identification of these serogroups is recognized as a key step to determining their prevalence, tracking the source of outbreaks, and for the development of new intervention strategies.

While several methods to detect STEC O157 have been developed, detection methods for non-O157 STEC O groups are limited, primarily because of an absence of distinctive features that enable easy differentiation of these serotypes. The development of PCR-based and related molecular approaches for the identification of STEC O groups (Valadez A M, DebRoy C et al., 2010. J. Food Prot. 74:228-239.; Fratamico et al., 2011. Foodborne Path. Dis. 8:601-7; DebRoy et al. 2011. Foodborne Path. Dis. 8:651-652), although promising, these PCR based approaches require specialized instrumentation and trained personnel, and therefore are not widely applicable outside of sophisticated laboratory settings.

In general embodiments, the invention provides for the rapid, sensitive, and specific identification and quantification of gram negative bacteria from a sample. The development of the methods has been described in detail in the examples section which follows. Briefly, the assay is based on an enzyme linked immunosorbant assays (ELISAs). The assay was able to detect and identify the top six non-O157 STEC O groups. The assays were tested against a total of 174 reference E. coli O groups, 60 E. coli clinical isolates, and 10 other bacterial species. The results showed that the assays are highly specific (98%-100%) with a limit of detection of 10-20 CFU of bacteria following enrichment and an analytical sensitivity comparable to extant commercially available immunoassays. Importantly, the newly developed assays enabled a reduction in time from more than 5 day using current protocols to less than 2 days. Taken together, these rapid assays have considerable utility for detecting STEC O groups in food and in other sources, and provide the basis for the rapid detection of other serogroups of E. coli as well as other Gram-negative pathogens.

According to one aspect of the invention the antigen is a characteristic component of a gram negative bacterium. In preferred embodiments, the antigen comprises a conserved region and a variable region which differs between serogroups both within the genus but also between the types of bacterium. This can be a part of the pathogen, such as a particular structural protein, or a product which the pathogen makes, such as a toxin. Bacterial toxins which can be tested include any gram negative toxin for example, Shiga toxin. Whole bacterial cells can also be detected using their particular antigenic molecules. Examples of other bacteria include: Citrobacter freundii, Enterobacter cloacae, Hafnia alvei, Klebsiella pneumonia, Proteus vulgaris, Salmonella enteritidis, Salmonella typhi, Serratia marcescens, Shigella boydii, Shigella flexneri, Neisseria gonorrhoeae, Neisseria meningitis, Moraxella catarrhalis, Acinetobacter baumannii. Examples of bacterial species comprise: Escherichia, Salmonella, Shigella, Enterobacteriaceae, Pseudomonas, Helicobacter, Legionella, Neisseria, Hemophilus, Salmonella, Serratia, Klebsiella, Chlamydiae, Cyanobacteria, Proteobacteria, Pseudomonadaceae, Moraxella, Vibrio, Aeromonas, Brucella, Francisella, Bordetella, Bartonella, Coxiella, Pasteurella, Mannheimia, Actinobacillus, Gardnerella, Spirochaetaceae, Leptospiraceae, Campylobacter, Spirillum, Streptobacillus, Bacteroidaceae, Acinetobacter, and the like. In other embodiments, the bacterium is a gram positive bacterium.

In another preferred embodiment, the specificity of the assay can allow the detection of the antigen from complex biological mixtures, such as for example, urine, serum, biopsies, tissue extracts, or food.

In a preferred embodiment, a method of detecting and quantifying variable antigens in a biological sample comprises coating a biological assay device with a first agent which specifically binds to epitopes in a conserved region of an antigen wherein the antigen comprises conserved and variable regions. The antigen is then added, preferably incubated to allow specific capture of the antigen. A second agent specific for the variable region epitopes is added which specifically binds to the captured or immobilized antigen. The second agent typically is labeled with an enzyme. The enzyme can be used in a colorimetric or fluorimetric reaction, for example, to produce a visually detectable product. Such visually detectable products are known in the art, and any suitable set can be used. Examples include: fluorescent, radioactive, chromatic, optical, and other physical or chemical labels.

In preferred embodiments, the antigen is a gram negative bacterial antigen. However, one of ordinary skill in the art could discern a multitude of antigens for detection. For example, lipid A or precursors molecules thereof and/or lipoteichoic acid or precursor molecules thereof. In one aspect, the antigen is derived from gram negative bacteria and comprises a conserved region of lipid A and a variable region. The variable region is, for example, an E. coli O antigen. In other embodiments, a conserved region is a variant of lipid A. In other embodiments, the conserved region is a synthetic molecule, for example, a peptide. The peptide can comprise one or more immunogenic epitopes or, for example, the molecule is a ligand detectable by its specific binding partner. The binding partner can be, for example, an antibody, aptamer, glycoprotein, protein, oligonucleotide and the like.

In general embodiments, the variable region is one or more antigens from Gram-negative bacteria of bacterial groups, families, genera or species comprising strains pathogenic for humans or animals like Enterobacteriaceae (Escherichia, especially E. coli, Salmonella, Shigella, Citrobacter, Edwardsiella, Enterobacter, Hafnia, Klebsiella, especially K. pneumoniae, Morganella, Proteus, Providencia, Serratia, Yersinia), Pseudomonadaceae (Pseudomonas, especially P. aeruginosa, Burkholderia, Stenotrophomonas, Shewanella, Sphingomonas, Comamonas), Neisseria, Moraxella, Vibrio, Aeromonas, Brucella, Francisella, Bordetella, Legionella, Bartonella, Coxiella, Haemophilus, Pasteurella, Mannheimia, Actinobacillus, Gardnerella, Spirochaetaceae (Treponema and Borrelia), Leptospiraceae, Campylobacter, Helicobacter, Spirillum, Streptobacillus, Bacteroidaceae (Bacteroides, Fusobacterium, Prevotella, Porphyromonas), Acinetobacter, especially A. baumanii. It is to be understood, that the methods embodied herein, can be applied to other organisms, such as for example, gram-positive bacteria, viruses, parasites, etc. In embodiments, the non-variable or conserved region can be, for example, lipid A and the variable antigen from an organism to be tested can be conjugated to the non-variable region. The term “non-variable” means that the molecule is of the same sequence or composition across species. Thus, for example if the organism is E. coli, then the lipid non-variable region will contain the same epitopes throughout the various E. coli serogroups.

In another preferred embodiment, wherein the first and second agents are antibodies or fragments thereof. In some aspects the antibodies are polyclonal, monoclonal, synthetic or combinations thereof.

Assays:

Depending on the device used, the assay can be modified to accommodate the format of the assay. For example, if the assay is conducted in, a 96-well plate, the appropriate dilutions and conditions can be adapted to suit the format. If a biochip or any high-throughput assay is used, the steps can be modified to accommodate the device, such as for example, printing of the first agent on the chip, or printing of the antigen on the chip. One of skill in the art would recognize the any modifications to be conducted. However, the principle remains the same.

In one preferred embodiment, the assay is an enzyme linked immunosorbent assay (ELISA) and the biological device is a multi-well plate. In such embodiments, a detectable label will be the use of an enzyme and substrate to produce the output. In other examples, the label can be radioactive, as in the case of a radioimmunoassay.

In other embodiments, the assay comprises immunoassays, lateral flow assays, biochip assays, protein assays, or high-throughput screening assays. For example, arrays have been designed as a miniaturization of familiar immunoassay methods such as ELISA and dot blotting, often utilizing fluorescent readout, and facilitated by robotics and high throughput detection systems to enable multiple assays to be carried out in parallel. Common physical supports include glass slides, silicon, microwells, nitrocellulose or PVDF membranes, and magnetic and other microbeads. While microdrops of protein delivered onto planar surfaces are widely used, related alternative architectures include CD centrifugation devices based on developments in microfluidics and specialized chip designs, such as engineered microchannels in a plate and tiny 3D posts on a silicon surface. Particles in suspension can also be used as the basis of arrays, providing they are coded for identification; systems include color coding for microbeads [Luminex, Bio-Rad] and semiconductor nanocrystals [QDots™, Quantum Dots], and barcoding for beads [ULTRAPLEX™, Smartbeads] and multimetal microrods [NANOBARCODES™ particles, Surromed]. Beads can also be assembled into planar arrays on semiconductor chips [LEAPS technology, BioArray Solutions].

In other embodiments, a first agent is attached to or immobilized on a solid support. In certain embodiments, the solid support is a bead (e.g., a colloidal particle, nanoparticle, latex bead, etc.), a flow path in a lateral flow immunoassay device (e.g., a porous membrane), a flow path in an analytical rotor, or a tube or well (e.g., in a plate suitable for an ELISA assay).

Thus in some aspects, the present invention provides a method for detecting the presence of an antigen, preferably simultaneous or parallel detection of multiple antigens, in a sample. The method includes providing a sample which optionally has been denatured and/or fragmented to generate a collection of soluble antigens (analytes); providing an array comprising a support having a plurality of discrete regions to which are bound a plurality of capture first agents, wherein each of the capture agents is bound to a different discrete region and wherein each of the capture agents is able to recognize and interact with a unique conserved region epitopes within the antigen; contacting the array of capture agents with the antigen; adding a second detecting agent which is specific for unique epitopes in the variable regions of the antigen and determining which discrete regions show specific binding to the sample, thereby detecting the presence of the antigen in a sample.

The capture agent array can be produced on any suitable solid surface, including silicon, plastic, glass, polymer, such as cellulose, polyacrylamide, nylon, polystyrene, polyvinyl chloride or polypropylene, ceramic, photoresist or rubber surface. Preferably, the silicon surface is a silicon dioxide or a silicon nitride surface. Also preferably, the array is made in a chip format. The solid surfaces may be in the form of tubes, beads, discs, silicon chips, microplates, polyvinylidene difluoride (PVDF) membrane, nitrocellulose membrane, nylon membrane, other purous membrane, non-porous membrane, e.g., plastic, polymer, perspex, silicon, amongst others, a plurality of polymeric pins, or a plurality of microtiter wells, or any other surface suitable for immobilizing proteins and/or conducting an immunoassay or other binding assay.

In some aspects, the first agent or the capture agent may be a protein, a peptide, an antibody, e.g., a single chain antibody, an artificial protein, an RNA or DNA aptamer, an allosteric ribozyme or a small molecule. Examples include, without limitation: nucleotides; nucleic acids including oligonucleotides, double stranded or single stranded nucleic acids (linear or circular), nucleic acid aptamers and ribozymes; PNA (peptide nucleic acids); proteins, including antibodies (such as monoclonal or recombinantly engineered antibodies or antibody fragments), T cell receptor and MHC complexes, lectins and scaffolded peptides; peptides; other naturally occurring polymers such as carbohydrates; artificial polymers, including plastibodies; small organic molecules such as drugs, metabolites and natural products; and the like.

In certain embodiments, the first or capture agents are immobilized, permanently or reversibly, on a solid support such as a bead, chip, or slide. The antigen and second binding agent can be added consecutively. Alternatively, the antigen and second agent are incubated to allow for specific binding prior to adding to the immobilized first agent or capture agent.

In other embodiments, the first agent, is attached to or immobilized on a substrate, such as a solid or semi-solid support. The attachment can be covalent or non-covalent, and can be facilitated by a moiety associated with the first agent that enables covalent or non-covalent binding, such as a moiety that has a high affinity to a component attached to the carrier, support or surface. For example, the first agent can be associated with a ligand, such as biotin, and the component associated with the surface can be a corresponding ligand receptor, such as avidin. The first agent can be attached to or immobilized on the substrate either prior to or after the addition of a sample containing antibody during an immunoassay.

In certain embodiments, the substrate is a bead, such as a colloidal particle (e.g., a colloidal nanoparticle made from gold, silver, platinum, copper, metal composites, other soft metals, core-shell structure particles, or hollow gold nanospheres) or other type of particle (e.g., a magnetic bead or a particle or nanoparticle comprising silica, latex, polystyrene, polycarbonate, polyacrylate, or PVDF). Such particles can comprise a label (e.g., a colorimetric, chemiluminescent, or fluorescent label) and can be useful for visualizing the location of the peptides during immunoassays. In certain embodiments, a terminal cysteine of a peptide, as an example of a first agent, is used to bind the peptide directly to the nanoparticles made from gold, silver, platinum, copper, metal composites, other soft metals, etc.

In certain embodiments, the substrate is a dot blot or a flow path in a lateral flow immunoassay device. For example, the first agent can be attached or immobilized on a porous membrane, such as a PVDF membrane (e.g., an Immobilon™ membrane), a nitrocellulose membrane, polyethylene membrane, nylon membrane, or a similar type of membrane.

In certain embodiments, the substrate or support is a flow path in an analytical rotor. In other embodiments, the substrate is a tube or a well, such as a well in a plate (e.g., a microtiter plate) suitable for use in an ELISA assay. Such substrates or support can comprise glass, cellulose-based materials, thermoplastic polymers, such as polyethylene, polypropylene, or polyester, sintered structures composed of particulate materials (e.g., glass or various thermoplastic polymers), or cast membrane film composed of nitrocellulose, nylon, polysulfone, or the like. A substrate can be sintered, fine particles of polyethylene, commonly known as porous polyethylene, for example, 0.2-15 micron porous polyethylene from Chromex Corporation (Albuquerque, N. Mex.). All of these substrate materials can be used in suitable shapes, such as films, sheets, or plates or they may be coated onto or bonded or laminated to appropriate inert carriers, such as paper, glass, plastic films, or fabrics. Suitable methods for immobilizing peptides on solid phases include ionic, hydrophobic, covalent interactions and the like.

In general embodiments, the substrate may be any surface or support upon which the first agent can be immobilized, including one or more of a solid support (e.g., glass such as a glass slide or a coated plate, silica, plastic or derivatized plastic, paramagnetic or non-magnetic metal), a semi-solid support (e.g., a polymeric material, a gel, agarose, or other matrix), and/or a porous support (e.g., a filter, a nylon or nitrocellulose membrane or other membrane). In some embodiments, synthetic polymers can be used as a substrate, including, e.g., polystyrene, polypropylene, polyglycidylmethacrylate, aminated or carboxylated polystyrenes, polyacrylamides, polyamides, polyvinylchlorides, and the like. In preferred embodiments, the substrate comprises a microtiter immunoassay plate or other surface suitable for use in an ELISA.

The surface of the substrate or support may be planar, curved, spherical, rod-like, pointed, wafer or wafer-like, or any suitable two-dimensional or three-dimensional shape on which the first agent may be immobilized, including, e.g., films, beads or microbeads, tubes or microtubes, wells or microtiter plate wells, microfibers, capillaries, a tissue culture dish, magnetic particles, pegs, pins, pin heads, strips, chips prepared by photolithography, etc. In some embodiments, the surface is UV-analyzable, e.g., UV-transparent.

Immobilization may be achieved in any number of ways, known in the art, described herein, and/or as can be developed. For example, immobilization may involve any technique resulting in direct and/or indirect association of an analyte (and its corresponding antagonist) with the substrate, including any means that at least temporarily prevents or hinders its release into a surrounding solution or other medium. The means can be by covalent bonding, non-covalent bonding, ionic bonding, electrostatic interactions, Hydrogen bonding, van der Waals forces, hydrophobic bonding, or a combination thereof. For example, immobilization can be mediated by chemical reaction where the substrate contains an active chemical group that forms a covalent bond with the first agent. For example, an aldehyde-modified support surface can react with amino groups in protein receptors; or amino-based support surface can react with oxidization-activated carbohydrate moieties in glycoprotein receptors; a support surface containing hydroxyl groups can react with bifunctional chemical reagents, such as N,N dissuccinimidyl carbonate (DSC), or N-hydroxysuccinimidyl chloroformate, to activate the hydroxyl groups and react with amino-containing receptors. In some embodiments, support surface of the substrate may comprise animated or carboxylated polystyrenes; polyacrylamides; polyamines; polyvinylchlorides, and the like. In still some embodiments, immobilization may utilize one or more binding-pairs to bind or otherwise attach a receptor to a substrate, including, but not limited to, an antigen-antibody binding pair, hapten/anti-hapten systems, a avidin-biotin binding pair; a streptavidin-biotin binding pair, a folic acid/folate binding pair; photoactivated coupling molecules, and/or double stranded oligonucleotides that selectively bind to proteins, e.g., transcriptional factors.

The first agent may be immobilized on the surface of the second reaction container or to beads within the second reaction container or added to the first reaction container (e.g. superparamagnetic polystyrene beads—Dynabeads M-450).

In other embodiments, the order of adding the first and second agents can be revered. Thus, for example, the second agent is immobilized on a substrate; the antigen to be tested is added followed by the first agent which may comprise a detectable label.

In one embodiment, the second agent is conjugated with a reporter molecule such as a fluorescent molecule or an enzyme, and used to detect the presence of bound antigen on the assay device (such as a chip or bead), in for example, a “sandwich” type assay in which one capture agent is immobilized on a support to capture the antigen, while a second, labeled agent also specific for the captured antigen is added to detect/quantitate the captured antigen. In other embodiments a labeled-antigen is used in a competitive binding assay to determine the amount of unlabeled antigen (from the sample) bound to the capture agent.

Conventional reporter molecules or labels may be used which are capable, alone or in concert with other compositions or compounds, of providing a detectable signal. Suitable labels include, but are not limited to, enzymes (e.g., HRP, β-galactosidase, etc.), fluorescent labels, radioactive labels, and metal-conjugated labels (e.g., colloidal gold-conjugated labels). Suitable detection methods include, e.g., detection of an agent which is tagged, directly or indirectly, with a colorimetric assay (e.g., for detection of HRP or beta-galactosidase activity), visual inspection using light microscopy, immunofluorescence microscopy, including confocal microscopy, or by flow cytometry (FACS), autoradiography (e.g., for detection of a radioactively labeled agent), electron microscopy, immunostaining, subcellular fractionation, or the like. In one embodiment, a radioactive element (e.g., a radioactive amino acid) is incorporated directly into a peptide chain; in another embodiment, a fluorescent label is associated with a peptide via biotin/avidin interaction, association with a fluorescein conjugated antibody, or the like. In one embodiment, a detectable specific binding partner for the antibody is added to the mixture. For example, the binding partner can be a detectable secondary antibody or other binding agent (e.g., protein A, protein G, protein L) which binds to the first antibody. This secondary antibody or other binding agent can be labeled, e.g., with a radioactive, enzymatic, fluorescent, luminescent, or other detectable label, such as an avidin/biotin system. In another embodiment, second agent is conjugated directly or indirectly (e.g. via biotin/avidin interaction) to an enzyme, such as horseradish peroxidase or alkaline phosphatase. In such embodiments, the detectable signal is produced by adding a substrate of the enzyme that produces a detectable signal, such as a chromogenic, fluorogenic, or chemiluminescent substrate.

It may also be desirable to couple more than one reporter group to a first or second agent, depending which agent is added first. In one embodiment, multiple reporter groups are coupled to one second agent molecule. In another embodiment, more than one type of reporter group may be coupled to one first agent. Regardless of the particular embodiment, first or second agents with more than one reporter group may be prepared in a variety of ways. For example, more than one reporter group may be coupled directly to a first or second detection agent, or linkers that provide multiple sites for attachment can be used.

A sample for use in the invention can refer to specimen material used for a given assay, reaction, run, trial, and/or experiment. For example, a sample may comprise an aliquot of the specimen material collected, up to and including the entire specimen. Samples may be crude samples or processed samples, e.g., obtained after various processing or preparation steps carried out on the original specimen. For example, various cell separation methods, e.g., magnetically activated cell sorting, may be applied to separate or enrich analytes of interest in a biological fluid, such as blood. A sample may also comprise a dilution of a specimen, e.g., diluted serum or dilutions of other complex and/or protein-rich mixtures. For example, in some embodiments, a specimen may be serially diluted to provide a number of serially-diluted samples for analysis. As used herein the terms assay, reaction, run, trial and/or experiment can be used interchangeably. Preferred embodiments of the present invention can be practiced using small starting amounts of analytes to yield quantifiable results.

Samples may be taken from an in vivo or in vitro source. Examples of in vivo sources include blood extracts, serum, cell extracts or homogenized tissue taken from a patient. Examples of in vitro sources include tissue culture extracts or growth media extracts from a variety of cell cultures including animal cell lines, hybridomas, yeasts and bacteria.

Analytes for use in the invention include but are not limited to e.g., small molecules such as small natural or synthetic organic molecules of up to 2000 Da, preferably 800 Da or less; peptidomimetics; inorganic molecules; drugs or pharmaceuticals; cytokines; toxins; macromolecular structures; metabolites; natural or modified substrates; steroids; enzymes; hormones; nucleic acids; proteins; receptors; peptides; glycoproteins; domains or motifs; amino acids; lectins; lipids; carbohydrates; sugars; polymers; tissues; cells; cell surface components; cellular components; subcellular organelles; whole or parts of microbes (such as pathogens, parasites, viruses, bacteria, fungi, and the like) from any known organism, more particularly any known virus, bacteria or fungus, in particular HIV, HCV, HBV, Influenza, Rhinovirus, Picornavirus, Poliovirus, Shiga, E. coli, Helicobacter sp., Enterobacter sp., Salmonella sp., Chlamydia sp., Enterococcus sp., Staphylococcus sp., Streptococcus sp., Bacillus sp., Neisseria sp. Candida sp., Cryptococcus sp., Aspergillus sp; biological and chemical warfare agents; and/or antibodies e.g. to one or more of the above described molecule, including IgG, IgA, IgM, IgD and IgE; structural or functional mimetics of the aforementioned or any combinations thereof.

Compositions:

In another preferred embodiment, a composition comprises an agent which specifically binds to conserved or variable epitopes of gram negative bacterial antigens. Antibodies raised against antigens are directed against epitopes in the variable region. The antibodies include antibodies from any species, including humans.

In one embodiment, the capture agent is an antibody or an antibody-like molecule (collectively “antibody”). Thus an antibody useful as capture agent may be a full length antibody or a fragment thereof, which includes an “antigen-binding portion” of an antibody. The term “antigen-binding portion,” as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H} are coded for by separate genes, they can be joined, using recombinant methods, by a synthetic linker that enables them to be made as a single protein chain in which the V_{L} and V_{H} regions pair to form monovalent molecules (known as single chain Fv (scFv). Such single chain antibodies are also intended to be encompassed within the term “antigen-binding portion” of an antibody. V_{H} and V_{L} can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule and use of a cysteine residue, a marker peptide and a C-terminal polyhistidine tag to make bivalent and biotinylated scFv molecules. Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques.

Antibodies may be polyclonal or monoclonal. The terms “monoclonal antibodies” and “monoclonal antibody composition,” as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody composition, typically displays a single binding affinity for a particular antigen with which it immunoreacts.

In embodiments, a composition comprises a chimeric molecule having at least two domains wherein a first domain comprises a predetermined epitope and at least one second domain comprising an antigen wherein the first and second domains are conjugated, covalently bound, fused or linked. For example, the predetermined epitope can be a peptide, lipid (e.g. lipid A), glycoprotein, nucleic acids etc., of known sequence. The antigen to be tested can be any desired antigen, such as for example, gram negative bacterial antigens. The two domains can be linked via any mechanism known in the art. For example, linker molecules, covalent bonding and the like. The variable antigens can thus be identified and quantified in an assay embodied herein. In embodiments, the composition can be used in assays such as, for example, an ELISA.

Conjugates can be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

Coupling can be accomplished by any chemical reaction that will bind the two molecules and retain their respective activities. This linkage can include many chemical mechanisms, for instance covalent binding, affinity binding, intercalation, coordinate binding and complexation. Covalent binding is also useful. Covalent binding can be achieved either by direct condensation of existing side chains or by the incorporation of external bridging molecules. Many bivalent or polyvalent linking agents are useful in coupling protein molecules, such as the disclosed antibodies, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (See Killen and Lindstrom, Jour. Immun. 133:1335-2549 (1984); Jansen et al., Immunological Reviews 62:185-216 (1982); and Vitetta et al., Science 238:1098 (1987)). Examples of useful linkers are described in the literature (See, for example, Ramakrishnan, S. et al., Cancer Res. 44:201-208 (1984) describing use of MBS (M-maleimidobenzoyl-N-hydroxysuccinimide ester). See also U.S. Pat. No. 5,030,719, describing use of halogenated acetyl hydrazide derivative coupled to an antibody by way of an oligopeptide linker. Useful linkers include: (i) EDC (1-ethyl-3-(3-dimethylamino-propyl)carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio)propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS(N-hydroxysulfo-succinimide: Pierce Chem. Co., Cat. #24510) conjugated to EDC.

The linkers described above can contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings when used in conjunction with sulfo-NHS, forms esters that are more resistant to hydrolysis than the carbodimide coupling reaction alone.

In one embodiment, a method of detecting and quantifying variable antigens in a biological sample comprising: coating a biological assay device with a first agent which specifically binds to the predetermined epitope; adding a composition comprising a chimeric molecule having at least two domains wherein a first domain comprises the predetermined epitope and at least one second domain comprising a variable antigenic region to be tested, wherein the first and second domains are fused or linked, and the first agent specifically binds to the epitope of the first domain; adding a second agent which specifically binds to the variable antigenic regions of the second domain.

In embodiments, the first domain comprises: a peptide, oligonucleotide, synthetic molecule, glycoprotein, lipids, carbohydrates, organic compounds, inorganic compounds or combinations thereof.

In embodiments, the second domain is an antigen to be tested and can comprise a single antigenic epitope or a plurality of varying antigenic epitopes. In some embodiments, the first domain is conjugated to attached to the second domain via fusion, a linker molecule, covalent bonds, carbohydrates, or combinations thereof.

In embodiments, the first and second agents comprise: antibodies or fragments thereof, aptamers, scaffolded peptides, nucleotides; nucleic acids; PNA (peptide nucleic acids); proteins; peptides; carbohydrates; artificial polymers; synthetic or natural molecules, organic or inorganic molecules.

Kits:

Kits are provided here, which comprise components in a package for ready usage in the assays according to the invention. Typically, written instructions to practice the methods of the invention will also be provided. The kits may include antibodies, media, purified samples of antigen for use as positive controls. Suitable buffers for diluting or reconstituting test samples, antibodies may also be provided. Some of the components may be provided in dry form, and may require reconstitution. The first agent may be prebound to the assay device. The surfaces may be divided up into any geometric shape or size. They may be provided in reaction vessels, such as in microtiter dishes. Thus in one embodiment, a kit for the detection, identification and quantification of bacterial antigens comprises a first agent which specifically binds to a conserved region epitope of the bacterial antigen and a second agent which specifically binds to a variable region epitope of the bacterial antigen. The kit may optionally comprise a detectable label.

In other embodiments, the substrates or surfaces of the biological device are pre-coated. In other embodiments the first or second binding agents are immobilized on a substrate.

Reagents for particular types of assays can also be provided in kits of the invention. Thus, the kits can include a population of beads (e.g., suitable for an agglutination assay or a lateral flow assay), or a plate (e.g., a plate suitable for an ELISA assay). In other embodiments, the kits comprise a device, such as a lateral flow immunoassay device, an analytical rotor, or an electrochemical, optical, or opto-electronic sensor. The population of beads, the plate, and the devices are useful for performing an immunoassay. F or example, they can be useful for detecting formation of a first agent-analyte-second agent complex.

In addition, the kits can include various diluents and buffers, labeled conjugates or other agents for the detection of specifically bound antigens or antibodies, and other signal-generating reagents, such as enzyme substrates, cofactors and chromogens. Other components of a kit can easily be determined by one of skill in the art. Such components may include coating reagents, polyclonal or monoclonal capture antibodies specific for an antigen or analyte to be tested, or a cocktail of two or more of the antibodies, purified or semi-purified extracts of these antigens as standards, monoclonal antibody detector antibodies, an anti-mouse, anti-dog, anti-chicken, or anti-human antibody with indicator molecule conjugated thereto, indicator charts for colorimetric comparisons, disposable gloves, decontamination instructions, applicator sticks or containers, a sample preparatory cup, etc. In one embodiment, a kit comprises buffers or other reagents appropriate for constituting a reaction medium allowing the formation of a peptide-antibody complex.

Such kits provide a convenient, efficient way for a clinical laboratory to diagnose infection by a pathogenic E. coli or other bacteria. Thus, in certain embodiments, the kits further comprise an instruction. For example, in certain embodiments, the kits comprise an instruction indicating how to use the compositions of the invention for detection of a particular analyte. In certain embodiments, the kits comprise an instruction indicating how to use a population of beads, a plate, or a device to serotype, for example, E. coli O antigen, or to identify and/or diagnose an infection.

The invention has been described in detail with reference to preferred embodiments thereof. However, it will be appreciated that those skilled in the art, upon consideration of this disclosure, may make modifications and improvements within the spirit and scope of the invention. The following non-limiting examples are illustrative of the invention.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

Embodiments of the invention may be practiced without the theoretical aspects presented. Moreover, any theoretical aspects are presented with the understanding that Applicants do not seek to be bound by the theory presented.

### Example 1

### Detection of Top Six Non-O157 Escherichia Coli Serogroups by ELISA

The objective of the present study was to develop a rapid enzyme linked immunosorbent assay (ELISA) for detecting the six major STEC O groups that can be adopted easily by diagnostic laboratories, food industries, regulatory agencies, and researchers. The results show that the ELISAs that are described herein were highly sensitive, specific and reduced the time for E. coli O antigen serotyping from 5-9 days to 2 days.

Materials and Methods

Bacterial Strains.

E. coli strains O26 (H311b) O45 (K42), O91 (H307b), O103 (H515b), O111 (Stoke W), O121 (39w), O145 (E1385) and a total of 174 other standard reference strains belonging to serogroups O1 through O181 except O31, O47, O72, O93, O94, O122 that are not designated (Orskov et al. 1977 Bacteriol. Rev. 41:667-710), and other clinical isolates (n=60) belonging to the six non-O157 STEC O groups were selected from the collection at the E. coli Reference Center (ECRC) at Penn State. Other bacterial species, including Citrobacter freundii, Enterobacter cloacae, Hafnia alvei, Klebsiella pneumonia, Proteus vulgaris, Salmonella enteritidis, Salmonella typhi, Serratia marcescens, Shigella boydii, and Shigella flexneri, were also used for specificity analysis.

Antibody Preparation.

Polyclonal antibodies were raised against heat-killed, whole cell preparations of standard reference E. coli strains belonging to serogroups O26, O45, O103, O111, O121, and O145. These were made as previously described (Muldoon et al. 2006). Antibody purification was conducted by SDIX (Newark, Del.) using proprietary protocols.

ELISA Plate Preparation.

Polystyrene microtiter plates were coated with 100 μl of mouse monoclonal antibody to lipid A (1 μg/ml) (Abcam, Cambridge, Mass.) in 0.05M carbonate buffer (pH 9.0) and incubated overnight at 4 C. The coating solution was removed and wells were washed twice with 200 μl of phosphate buffered saline (PBS) and filled with 200 μl of blocking solution (5% dry milk in 0.01 M PBS with 0.05% Tween 20) and incubated at 4° C. overnight, following which the wells were washed in distilled water three times, and stored at 4° C. until use.

Antigen preparation. Isolates of E. coli serogroups O26, O45, O103, O111, O121, and O145 were grown in 5 ml Veal Infusion Broth (VIB) (Difco, Sparks, Md.) overnight at 37° C. The cell density of the culture was adjusted to 1 OD₆₀₀ with VIB and 1 ml of the resulting material was centrifuged at 6,000×g for 10 min and the cell pellet re-suspended in 1 ml of PBS and serially diluted in PBS for use in plate counts. Culture samples (1 ml) containing 10⁶ CFU/ml of bacterial cells were boiled for 1 h at 100° C., and 100 μl of the heat inactivated bacterial antigen was used for ELISAs. Antigens from other E. coli serogroups (reference standard strains belonging to O1 through O181 serogroups) and other bacteria were also prepared using the same procedure.

ELISA.

Antigens (100 μl) of each of the STEC O groups were dispensed in triplicate wells in microtiter plates coated with antibodies to lipid A that were prepared earlier, and incubated with shaking at 37° C. for 1 h. The solution in each well was removed and the wells were washed four times with 200 μl of distilled water. Antibodies prepared against each of the STEC O groups were diluted 1000-fold with 0.01 M PBS and 0.05% Tween 20 containing 5% dry milk. Diluted antibodies (100 μl) were added to each well containing the antigens. The plates were incubated for 1 h at 37° C. The solution in each well was removed and the wells washed four times with 200 μl of distilled water. Goat anti-rabbit IgG peroxidase conjugate (100 μl at 1:3000 dilution) (Sigma-Aldrich, Mo.) prepared in 0.01 M PBS, 0.05% Tween 20 and 5% dry milk was added to each well and the plates were incubated for 1 h at 37° C. Solutions in each well were removed and the wells were washed four times with 200 μl of distilled water. Finally, 100 μl of 1-step ultra TMB (Fisher Scientific) was added to each well and the plates were incubated for 15 minutes at room temperature. An equal volume of stopping solution (2 M H₂SO₄) was added and optical density was read at 450 nm using a plate reader (Biotek Instruments, Winooski, Vt.). Wells containing all components of the assay but without antigen, served as negative controls for determining the background noise. Background to signal ratio of 1:10 was considered as positive signal. All background readings were subtracted and averages of triplicate readings are presented. Since binding of antigens to polystyrene plates may vary, reproducibility of the assays were tested in triplicate using polystyrene plates obtained from different sources.

Sensitivity and Specificity of the Assays.

Sensitivity of the assays was determined by titrating with ten-fold dilutions of antigen prepared from a known density of bacterial cells. Clinical or analytical sensitivity, defined as the proportion of samples that test positive with the screening tests, were also determined. Specificity of the assays, defined as the proportion of reference strains that test negative with the screening tests, were determined by ELISAs for each O group against a total of 60 clinical isolates, 10 for each of the six STEC O groups and reference standard E. coli strains belonging to O1 to O181 serogroups and 10 other bacterial species belonging to Enterobacteriaceae.

Detecting STEC O Groups in Ground Beef Samples.

Ground beef (25 g) purchased from a local store was individually spiked with 1-10 CFU of 6 strains of each of the six STEC O groups (n=36) in 20 ml Tryptic Soy Broth (TSB) media. E. coli was enriched from meat samples as previously reported with slight modifications (Valadez et al. 2010, J. Food Prot. 74:228-239; Fratamico et al. 2011, Foodborne Path. Dis. 8:601-7). All samples were pre-enriched by incubating static for 6 h at 37° C. in the presence of vancomycin (16 mg/L). After the pre-enrichment step, bile salts (1.5 g/ml), rifampicin (2.0 mg/L), and potassium tellurite (1.0 mg/L) were added, and incubation was continued for 18 h at 42° C. with shaking Mock-inoculated ground beef samples were enriched similarly and served as the negative control. Following enrichment, the sample was centrifuged at low speed (400×g) and 1 ml of the supernatant containing the bacteria was centrifuged at 6000×g for 10 min. The cell pellet was re-suspended in 1 ml of PBS, boiled for 1 h at 100° C. and 100 μl of the antigen preparation was used with the ELISAs to determine the STEC O group. Real ground beef samples, that were known to carry the STEC O groups, were similarly tested.

Anti-Lipid A Binding Assay.

Antigens (1 ml) from E. coli O groups described above were mixed individually with 5 μg of anti-lipid A monoclonal antibody and incubated for 1 h at room temperature. After incubation, 100 μl of this antigen-antibody mixture was dispensed in triplicate in wells of a microtiter plate. Antigen (100 μl) at the same dilution but without the addition of anti-lipid A antibody served as a negative control.

Statistical Analysis.

The Student t test was used to examine the statistical significance of the assays conducted in triplicate.

Results and Discussion

O antigens, a part of the lipopolysaccharide (LPS) complex on the outer membrane of the bacteria, are composed of repeat units of oligosaccharides (O unit). The sugar residues in the O antigen vary considerably and differ in their arrangement and linkage between and within the O units making it the most variable region of the cell. The O antigens are highly immunogenic and responsible for stimulating innate immunity. Variability amongst the O groups provides the basis for serotyping and classification of E. coli. Conventional serotyping for O group identification is based on agglutination reactions that still remains one of the most comprehensive and simple method for testing O groups. The antisera are produced by immunization of rabbits with different O group reference strains (Orskov F, and Orskov I. 1984. Meth. Microbiol. 14:43-112; Orskov I, et al., 1977. Bacteriol. Rev. 41:667-710.) and contain antibodies against the O group injected. Agglutination that develops using the specific O group antisera is considered a positive O antigen reaction. However, if the organism is capsulated or rough and does not carry LPS, the agglutination reaction fails. In addition, there may be cross reactions with other O groups exhibiting equivocal results.

O antigens are also responsible for virulence of the organism. Certain O groups are more virulent than others. It has been shown that specific clones with certain serotypes are associated with definite diseases (Orskov et al. 1976, Med. Microbiol. Immunol. 162:73-80; Achtman and Plushke, 1986, Ann. Rev. Microbiol. 40:185-210). Therefore, detection of serogroups of E. coli is essential for determining pathogenicity, tracking the source in outbreaks and for epidemiological studies. Twenty to 70% of STEC infections worldwide are due to non-0157 STEC (WHO, 1998) and of the 81 serotypes identified, 71% of the isolates recovered belong to six serogroups (O26, O45, O111, O103, O121, and O145) (Brooks et al. 2005 J. Infect Dis. 192:190-198; WHO 1998 Zoonotic non-O157 Shiga toxin-producing Escherichia coli (STEC). Report of a WHO Scientific Working Group Meeting, 23 to 26 Jun. 1998, Berlin, Germany.; Fratamico et al. 2011 Foodborne Path. Dis. 8:601-7)

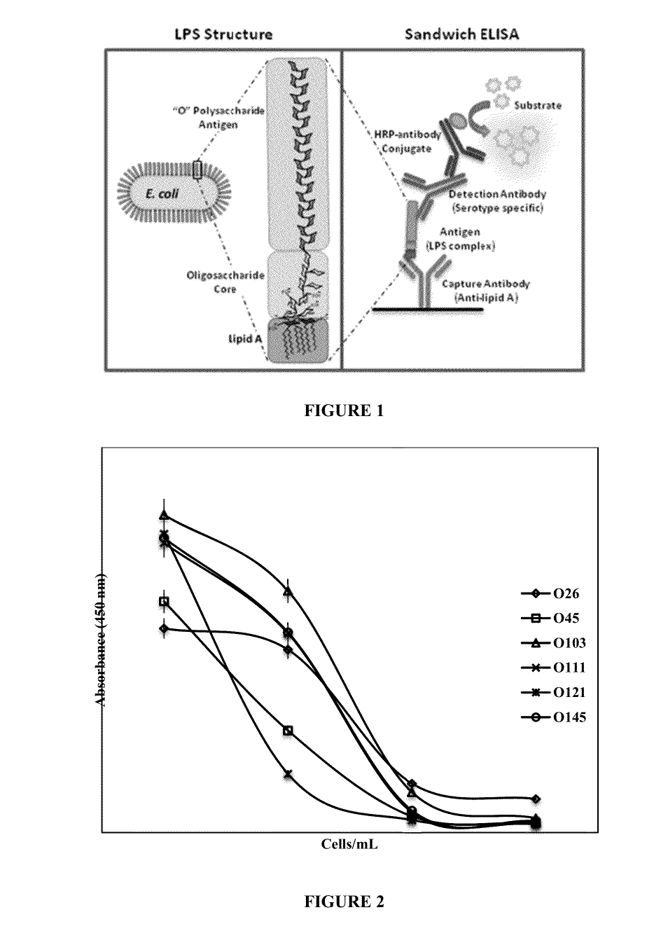

The present study describes a sandwich ELISA utilizing antigenic determinants in the LPS and O antigens to detect the six prevalent non-O157 STEC O serogroups. The wells of the microtiter plate were coated with antibodies against lipid A and the antigens produced by boiling E. coli were then added. The complex is detected by O specific detection antibodies as depicted in FIG. 1. The assays can be easily conducted without any sophisticated equipment. The ELISA assays were sensitive and could determine the O groups unequivocally at 10⁵CFU/ml concentrations (FIG. 2). Serogroup O103 exhibited the highest signal followed by O111, O145, O26, O45 and O121 (Table 1).

TABLE 1
Limit of detection of E. coli O groups by ELISAs

| | Aerobic Plate Count | Range of OD₄₅₀ | Background OD₄₅₀ |
|---|---|---|---|
| Serogroup | (CFU/ml) | in ELISA | in ELISA |
| O26 | 3.80 × 10⁵ | 0.407-0.479 | 0.201-0.258^{a} |
| O45 | 2.86 × 10⁵ | 0.235-0.328 | 0.084-0.098 |
| O103 | 2.37 × 10⁵ | 0.310-0.376 | 0.070-0.082 |
| O111 | 2.33 × 10⁵ | 0.286-0.353 | 0.085-0.113 |
| O121 | 1.05 × 10⁶ | 0.318-0.364 | 0.071-0.089 |
| O145 | 2.60 × 10⁵ | 0.313-0.490 | 0.077-0.100 |

The background OD₄₅₀ ranged from 0.071 to 0.113. An OD₄₅₀ of 0.2 above the background was considered positive.
^{a}Antibodies to serogroup O26 always provided a higher background.

The analytical or clinical sensitivity for each of the respective ELISAs was found to be 100% (10/10) for all of the O groups except O111 which displayed 90% (9/10) (Table 2).

TABLE 2
Specificity of antibodies tested. Cross reactivity of antibodies to top six
STEC serogroups tested against reference strains, non-E coli strains and clinical isolates.

| Strains tested | O26 | O45 | O103 | O111 | O121 | O145 |
|---|---|---|---|---|---|---|
| Standard Reference strains | Positive for | Positive for | Negative | Negative | Negative | Positive for |
| for serogroups O1 to O181 | O146, O156, | O146, O153, | for all | for all | for all | O150, O151 |
| except for the target | O167, O168 | O156, O168 | | | | |
| O group (n = 173) | | | | | | |
| Clinical isolates | Negative | Negative | Negative | Negative | Negative | Negative |
| 10 strains for each non- | for all | for all | for all | for all | for all | for all |
| targat top six STEC O | | | | | | |
| groups (n = 5 × 10 = 50) | | | | | | |
| Non-E. coli strains | Negative | Negative | Negative | Negative | Negative | Negative |
| (n = 10) | for all | for all | for all | for all | for all | for all |
| Total no. tested (n = 233) | Negative for | Negative for | Negative | Negative | Negative | Negative for |
| | 229 strains | 229 strains | for all | for all | for all | 231 strains |
| Specificity (%) | 98.2% | 98.2% | 100% | 100% | 100% | 99.14% |

Positive: OD₄₅₀ >0.2;
Negative: OD₄₅₀ <0.2.
All experiments were conducted using enriched culture at 10⁶-10⁷ CFU/ml concentrations.

The limit of detection was established to be 10-20 CFU for each target organism following enrichment. The sensitivity of the assay was 10⁶ cells/ml for all of the O groups.

Specificity of the ELISA for each O group was determined against designated E. coli reference standard strains belonging to O1 through O181 except the non-designated O groups. Among the six STEC O groups tested, antibodies against O103, O111, and O121 did not cross-react with any other E. coli serogroups. However, the ELISA for O145 was found to cross-react with serogroups O148, O150, O151, and O164, the O45 ELISA with serogroups O140, O146, O153, O156, and O168 and the O26 ELISA with O146, O156, O167 and O168. There was no cross-reactivity between the clinical isolates tested belonging to the 6 o groups and other bacteria species tested. While the specificity of the ELISA was 100% for O103, O111, and O121 O groups, the assays were 98% specific for O26, O45 and O145.

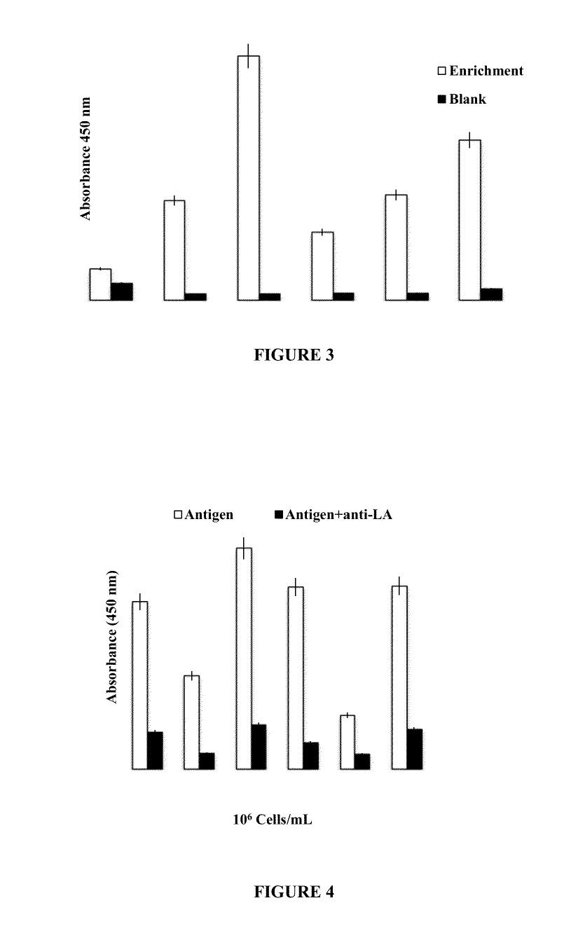

To validate the ELISAs in ground beef samples, 6 clinical isolates, including the reference strains, belonging to each of the six STEC O groups, were inoculated individually in ground beef at 1-10 CFU. The samples were enriched overnight and antigens were prepared as described. Un-inoculated ground beef served as a negative control. All assays exhibited 100% accuracy (FIG. 3). Real ground beef, that carried STEC O groups, also tested accurately using the ELISAs.

A competitive ELISA was performed to verify whether the lipid A component of LPS was indeed responsible for binding to antibodies against lipid A. To allow blocking of lipid A binding sites, antigens prepared from all six STEC O groups were mixed with anti-lipid A antibodies and incubated for 1 h before dispensing into the wells coated with anti-lipid A antibodies. Antigen preparations incubated with anti-lipid A antibody was found to bind only 10% of respective controls where the lipid A component of LPS complex was blocked by antibodies (FIG. 4) indicating that the lipid A component of the antigens was indeed responsible for binding to the anti-lipid A antibody coated on the wells of the microtiter plate.

The ELISAs described here for the six non-O157 STEC O groups are highly specific and sensitive and may be used in conjunction with assays for detecting shiga toxins and intimin in STEC serogroup positive samples to identify the potentially pathogenic strains.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

The Abstract of the Disclosure is provided to allow the reader to quickly ascertain the nature of the technical disclosure. It is submitted with the understanding that it will not be used to interpret or limit the scope or meaning of the following claims.

What is claimed:

1. A method of detecting and quantifying variable antigens in a biological sample comprising:
contacting a biological assay substrate with a first agent which specifically binds to epitopes in a conserved region of an antigen wherein the antigen comprises conserved and variable regions;
adding the antigen to a biological assay device wherein the agent specifically binds to the epitopes in the conserved region of the antigen;
adding a second agent which specifically binds to the variable region of the antigen, and;
detecting and quantifying variable antigens in a biological sample.

2. The method of claim 1, wherein the antigen is isolated from gram negative bacteria.

3. The method of claim 2, wherein the antigen comprises a conserved region of lipid A and a variable region.

4. The method of claim 1, wherein the second agent comprises a detectable label.

5. The method of claim 1, wherein the first and second agents comprise: antibodies or fragments thereof, aptamers, scaffold peptides, nucleotides; nucleic acids; PNA (peptide nucleic acids); proteins; peptides; carbohydrates; artificial polymers; synthetic or natural molecules, organic or inorganic molecules.

6. The method of claim 5, wherein the antibodies comprise: polyclonal, monoclonal, synthetic, an Fab fragment, an F(ab′)₂ fragment, an Fd fragment, an Fv fragment, a dAb fragment, an isolated complementarity determining region (CDR), a single chain antibody (scFv), or combinations thereof.

7. The method of claim 1, wherein the concentration of detectable antigen is at least about 0.0001 CFU/ml.

8. The method of claim 1, wherein the assay comprises immunoassays, lateral flow assays, biochip assays, protein assays, or high-throughput screening assays.

9. The method of claim 1, wherein the biological assay substrate comprises tubes, cylinders, beads, discs, silicon chips, microplates, polyvinylidene difluoride (PVDF) membrane, nitrocellulose membrane, nylon membrane, porous membranes, non-porous membranes, plastic, polymer, silicon, polymeric pins, a plurality of microtiter wells, or any combinations thereof.

10. A composition comprising a chimeric molecule having at least two domains wherein a first domain comprises a predetermined epitope and at least one second domain comprising an antigen wherein the first and second domains are fused or linked.

11. The composition of claim 10, wherein the antigen comprises variable epitopes of gram negative bacterial antigens.

12. A kit for the detection, identification and quantification of bacterial antigens comprising a first agent which specifically binds to a conserved region epitope of the bacterial antigen, a second agent which specifically binds to a variable region epitope of the bacterial antigen.

13. The kit of claim 12, further comprising a detectable label.

14. A method of detecting and quantifying variable antigens in a biological sample comprising:
contacting a biological assay substrate with an antigen or first agent wherein the antigen comprises conserved and variable regions, or immobilizing the antigen or first agent on surfaces of a biological device, wherein the first agent binds to the conserved regions of the antigen;
adding a second agent which specifically binds to the variable regions of the antigen, and;
detecting and quantifying variable antigens in a biological sample.

15. The method of claim 14, wherein the antigen is derived from gram negative bacteria.

16. The method of claim 14, wherein the first or second agents comprise a detectable label.

17. The method of claim 14, wherein the agent comprises: antibodies or fragments thereof, aptamers, scaffolded peptides, nucleotides; nucleic acids; PNA (peptide nucleic acids); proteins; peptides; carbohydrates; artificial polymers; synthetic or natural molecules, organic or inorganic molecules.

18. The method of claim 14, wherein the antibodies are polyclonal, monoclonal, synthetic, an Fab fragment, an F(ab′)₂ fragment, an Fd fragment, an Fv fragment, a dAb fragment, an isolated complementarity determining region (CDR), a single chain antibody (scFv), or combinations thereof.

19. The method of claim 14, wherein the conserved region of the antigen is immobilized on biological device surfaces, the surfaces comprising: silicon, plastic, glass, polymer, cellulose, polyacrylamide, nylon, polystyrene, polyvinyl chloride or polypropylene, ceramic, photoresist or rubber surface, silicon dioxide or a silicon nitride.

20. The method of claim 14, wherein a biological assay comprises immunoassays, lateral flow assays, biochip assays, protein assays, or high-throughput screening assays.

21. The method of claim 14, wherein the biological device comprises tubes, beads, discs. silicon chips, microplates, polyvinylidene difluoride (PVDF) membrane, nitrocellulose membrane, nylon membrane, porous membranes, non-porous membranes, plastic, polymer, silicon, polymeric pins, a plurality of microtiter wells, or any combinations thereof.

22. The method of claim 14, wherein the antigen is immobilized on a pre-treated surface, comprising: a protein, a peptide, an antibody, an artificial protein, an RNA or DNA aptamer, an allosteric ribozyme, nucleic acid, organic or inorganic molecule, carbohydrate or a small molecule.

23. A method of detecting and quantifying variable antigens in a biological sample comprising:
contacting a biological assay substrate with a first agent which specifically binds a predetermined epitope;
adding a composition comprising a chimeric molecule having at least two domains wherein a first domain comprises the predetermined epitope and at least one second domain comprising a variable antigenic region to be tested, wherein the first and second domains are fused or linked, and the first agent specifically binds to the epitope of the first domain;
adding a second agent which specifically binds to the variable antigenic regions of the second domain, and;
detecting and quantifying variable antigens in a biological sample.

24. The method of claim 23, wherein the first domain comprises: a peptide, oligonucleotide, synthetic molecule, glycoprotein, carbohydrates, organic compounds, inorganic compounds or combinations thereof.

25. The method of claim 23, wherein the second domain is an antigen to be tested.

26. The method of claim 23, the first domain is conjugated or attached to the second domain via fusion, a linker molecule, covalent bonds, carbohydrates, or combinations thereof.

27. The method of claim 23, wherein the first and second agents comprise: antibodies or fragments thereof, aptamers, scaffolded peptides, nucleotides; nucleic acids; PNA (peptide nucleic acids); proteins; peptides; carbohydrates; artificial polymers; synthetic or natural molecules, organic or inorganic molecules.

28. The method of claim 27, wherein the antibodies are polyclonal, monoclonal, synthetic, an Fab fragment, an F(ab′)₂ fragment, an Fd fragment, an Fv fragment, a dAb fragment, an isolated complementarity determining region (CDR), a single chain antibody (scFv), or combinations thereof.

29. The method of claim 27, wherein the second agent comprises a detectable label.

30. The method of claim 23, wherein the first agent is immobilized on a biological device surface, the surface comprising: silicon, plastic, glass, polymer, cellulose, polyacrylamide, nylon, polystyrene, polyvinyl chloride or polypropylene, ceramic, photoresist or rubber surface, silicon dioxide or a silicon nitride.

31. The method of claim 30, wherein the biological device comprises tubes, beads, discs. silicon chips, microplates, polyvinylidene difluoride (PVDF) membrane, nitrocellulose membrane, nylon membrane, porous membranes, non-porous membranes, plastic, polymer, silicon, polymeric pins, a plurality of microtiter wells, or any combinations thereof.

32. The method of claim 30, wherein the first agent or antigen is immobilized on an optionally pre-treated surface, comprising: a protein, a peptide, an antibody, an artificial protein, an RNA or DNA aptamer, an allosteric ribozyme, nucleic acid, organic or inorganic molecule, carbohydrate or a small molecule.
